(19) United States
(12) Patent Application Publication
(10) Pub. No.: US 2018/0078407 A1
(43) Pub. Date: Mar. 22, 2018

(54) SYSTEM, DEVICE AND METHOD FOR TREATMENT OF ENDOMETRIOSIS

(71) Applicant: BOSTON SCIENTIFIC SCIMED, INC., Maple Grove, MN (US)

(72) Inventors: Jai SHETAKE, Santa Clarita, CA (US); Bryan Allen CLARK, Forest Lake, MN (US); Stephen B. RUBLE, Lino Lakes, MN (US); Timothy Paul HARRAH, Cambridge, MA (US); David J. TERNES, Roseville, MN (US)

(21) Appl. No.: 15/700,993

(22) Filed: Sep. 11, 2017

Related U.S. Application Data
(60) Provisional application No. 62/396,719, filed on Sep. 19, 2016.

(51) Int. Cl. A61F 6/24; A61F 6/20

(52) U.S. Cl. CPC A61F 6/24; A61F 6/208

(57) ABSTRACT

A system for preventing retrograde flow in the fallopian tubes including a closure device comprising an elongated body, a channel extending therethrough, and a valve coupled to a proximal end of the body and open to the channel, the valve configured to only allow fluid to flow proximally therethrough from the channel. The system also includes a grasping element to secure the closure device within the fallopian tube comprising a plurality of tines, the tines including sharpened ends configured to penetrate the wall of the fallopian tube.

### PRIORITY INFORMATION

The present disclosure claims priority to U.S. Provisional Patent Application Ser. No. 62/396,719 filed Sep. 19, 2016; the disclosure of which is incorporated herewith by reference.

### BACKGROUND

Endometriosis (EM) is often misdiagnosed and/or untreated. Current inventions for EM include pharmacology and surgery. Pharmacologic interventions provide limited, short-term relief for pain associated with EM. Severe cases of EM often require surgery such as electric ablation of the endometrium or hysterectomies, which have drastic side effects such as reduction or elimination of the chances of pregnancy. One cause of EM is retrograde flow in which menstrual blood including endometrial cells flows backward into the fallopian tubes and into the pelvic cavity instead of out of the body. These cells can then adhere to the walls of pelvic organs where they thicken and can cause bleeding.

### SUMMARY

The present disclosure relates to a fallopian tube closure device, comprising an elongated body extending from a proximal end to a distal end and including a channel extending therethrough; and a valve coupled to a proximal end of the body and open to the channel, the valve configured to only allow fluid to flow proximally therethrough from the channel.

In an embodiment, the valve is a slit valve.

In an embodiment, the device further includes a tissue grasping element coupled to a distal end of the body.

In an embodiment, the grasping element comprises a plurality of tines, the tines including sharpened ends configured to penetrate the wall of the fallopian tube.

In an embodiment, the device further includes a snare element extending proximally from the body and configured to allow easy removal of the closure device.

In an embodiment, the snare element is configured as a cage extending about the length of the body.

In an embodiment, the grasping element is formed of Nitinol or another shape memory material.

In an embodiment, the elongated body is expandable from a low-profile insertion configuration to an expanded deployed configuration.

In an embodiment, the device further includes a delivery sheath extending between a proximal end and a distal end, wherein the body is slidably received within the delivery sheath, the elongated body sized and configured to fit in the sheath in a insertion configuration.

The present disclosure also relates to a system for preventing retrograde flow through the fallopian tubes comprising an elongated closure device extending from a proximal end to a distal end, the device sized and shaped to be inserted into the fallopian tube and an expansion device including an opening extending therethrough and configured to be inserted into the cervix such that the opening aligns with a cervix opening to be treated, the opening having a diameter greater than a diameter of the cervix opening to be treated.

In an embodiment, the body has a channel extending therethrough.

In an embodiment, the system further includes a valve coupled to a proximal end of the body and open to the channel, the valve configured to only allow fluid to flow proximally therethrough from the channel.

In an embodiment, the system further includes a tissue grasping element coupled to a distal end of the body.

In an embodiment, the grasping element comprises a plurality of tines, the tines including sharpened ends configured to penetrate the wall of the fallopian tube.

In an embodiment, the system further includes a snare element extending proximally from the body and configured to allow easy removal of the closure device.

The present disclosure also relates to a method for closing a fallopian tube comprising inserting a closure device into the fallopian tube, the closure device extending from a proximal end to a distal end and including a channel extending therethrough, and securing the closure device to the fallopian tube via one of friction fit and a grasping element, wherein the closure device further comprises a valve coupled to a proximal end thereof and open to the channel, the valve configured to only allow fluid to flow proximally therethrough from the channel.

In an embodiment, the grasping element comprises a plurality of tines, the tines including a sharpened end configured to penetrate the wall of the fallopian tube.

In an embodiment, the method further includes inserting a flexible delivery sheath into the lumen of the fallopian tube, the delivery sheath extending between a proximal end and a distal end and including a channel extending therethrough, the closure device is sized and shaped to fit within the sheath in a collapse configuration.

In an embodiment, the method further includes withdrawing the delivery sheath from the fallopian tube, wherein proximal movement of the delivery sheath moves the closure device proximally so that the grasping element penetrates a wall of the fallopian tube.

In an embodiment, the closure device includes a snare element extending proximally from the body and configured to allow easy removal of the closure device.

### BRIEF DESCRIPTION

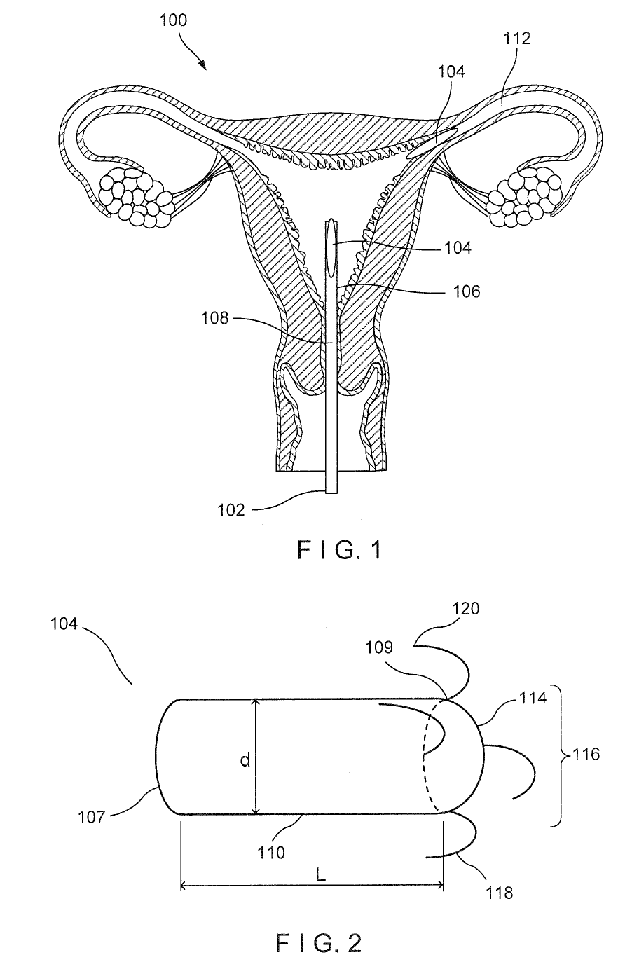

FIG. 1 shows a partially cross-sectional view of a device for closing or blocking a fallopian tube according to an exemplary embodiment of the present disclosure;

FIG. 2 shows a partially cross-sectional view of a closure device of the system of FIG. 1 according to an exemplary embodiment of the present disclosure;

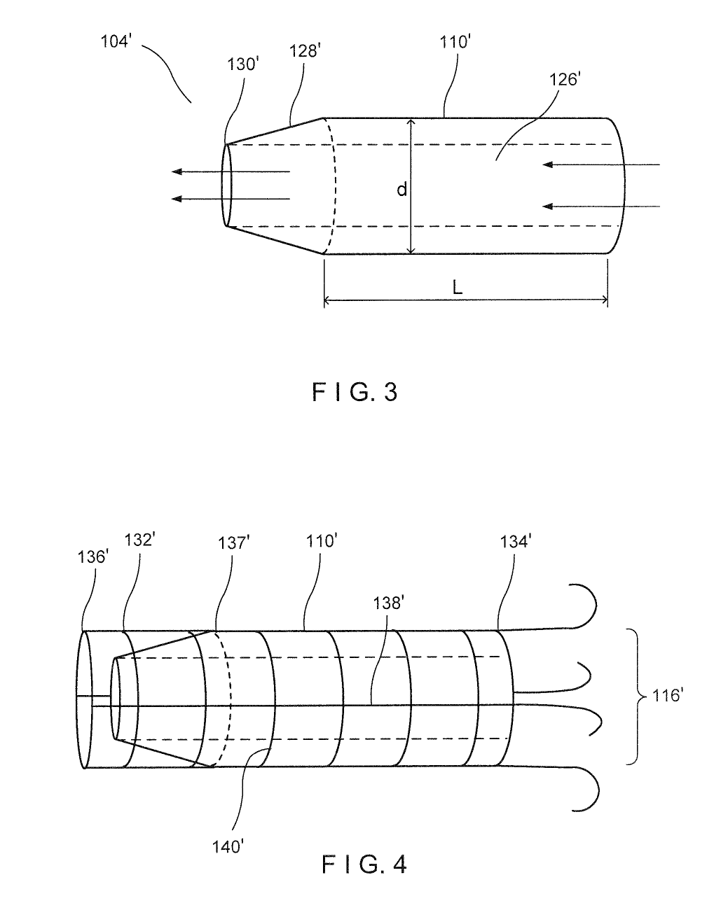

FIG. 3 shows a partially cross-sectional view of a closure device of the system of FIG. 1 according to a second exemplary embodiment of the present disclosure;

FIG. 4 shows a partially cross-sectional view of a closure device of the system of FIG. 1 according to an exemplary embodiment of the present disclosure;

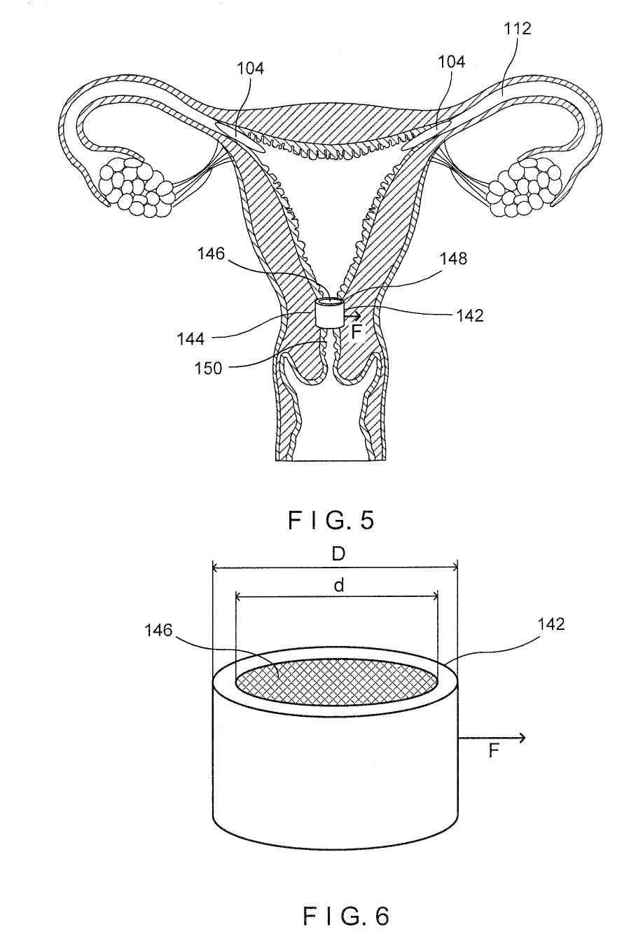

FIG. 5 shows a side view of an expansion device of the system of FIG. 1 according to an exemplary embodiment of the present disclosure;

FIG. 6 shows another side view of the expansion device of the system of FIG. 1 according to an exemplary embodiment of the present disclosure;

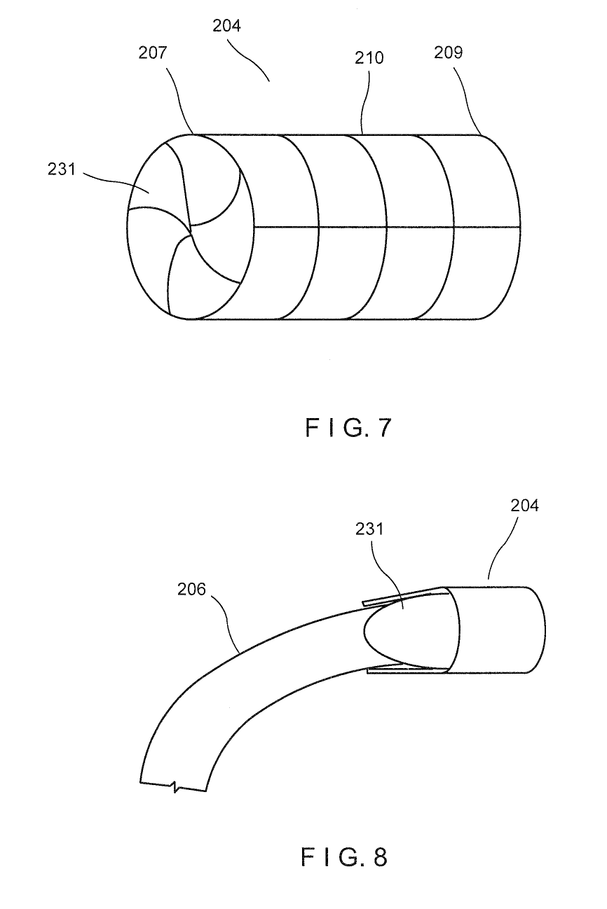

FIG. 7 shows a side view of a method for closing or blocking a fallopian tube according to a third exemplary embodiment of the present disclosure;

FIG. 8 shows a side view of a method for closing or blocking a fallopian tube according to the embodiment of FIG. 7;

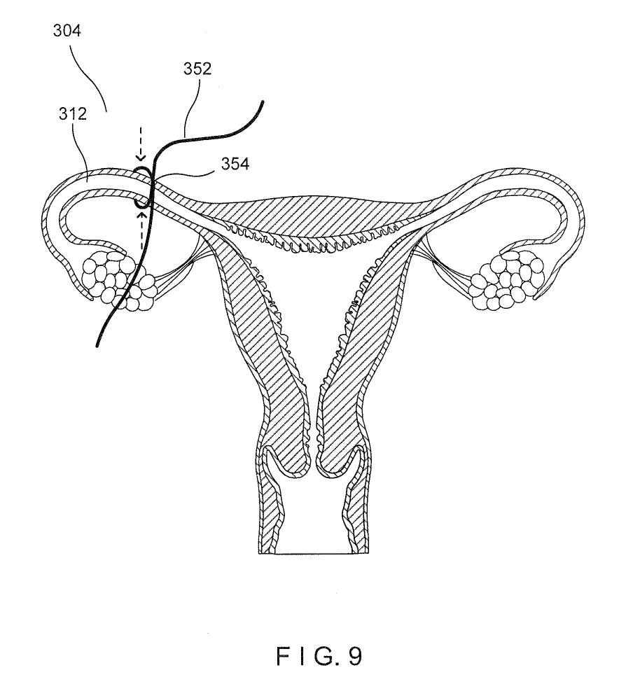

FIG. 9 shows a side view of a method for closing or blocking a fallopian tube according to a fourth exemplary embodiment of the present disclosure; and

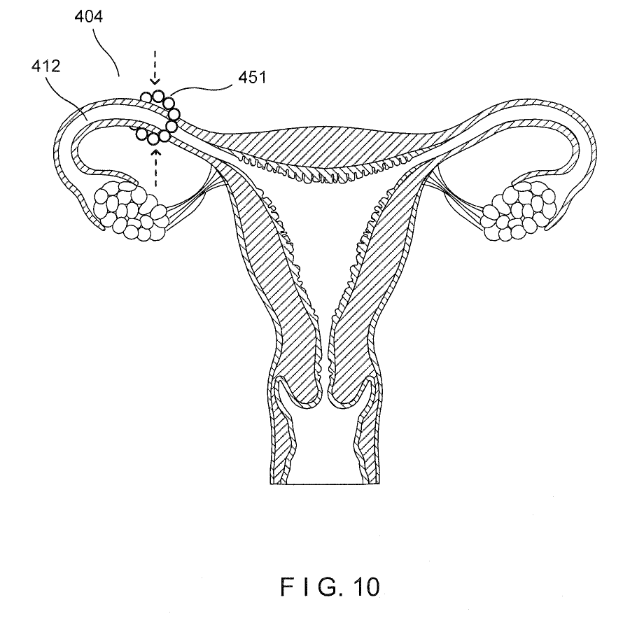

FIG. 10. shows a side view of a method for closing or blocking a fallopian tube according to a fifth exemplary embodiment of the present disclosure.

### DETAILED DESCRIPTION

The present disclosure may be further understood with reference to the appended drawings and the following description, wherein like elements are referred to with the same reference numerals. The present disclosure relates to devices and methods for closing or blocking an internal tubular member and, more particularly, relates to devices for closure or blocking of the fallopian tubes. It should be noted that the terms “proximal” and “distal,” as used herein, are intended to refer to a direction toward (proximal) and away from (distal) a user of the device (e.g. physician).

As shown in FIG. 1, a system 100 for controlling retrograde flow comprises a delivery element 102 within which a closure device 104 is slidably received therein. The delivery element 102 according to this embodiment is sized, for example, to pass through the working channel of an endoscope for delivery to a target site within a living body. As would be understood by those skilled in the art, the delivery element 102 is preferably sufficiently flexible while the closure device 104 is received therein to pass through a tortuous path through, for example, a natural body lumen without undue trauma to tissue along and adjacent to the lumen or damage to the closure device 104. For example, the delivery element 102 may have a flexibly sufficient to permit the delivery element 102 to be slidably inserted through a working channel of a device such as a flexible endoscope and to pass through any bending radii that these devices might achieve. The delivery element 102 may be formed as a flexible sheath 106 defining an internal lumen 108 within which the closure device 104 is slidably received. In an exemplary embodiment, the delivery element 102 is formed as a sheath 106 of polyether ether ketone (PEEK) having an outer diameter of less than 1 cm, preferably less than 5 mm, and ideally less than 1 mm at the distal end. However, as would be understood by those skilled in the art, other materials and sizes may be used. The delivery element 102 extends between a distal end, within which the closure device 104 is received, and a proximal end including handle (not shown) that, during use, remains outside the body accessible to a user. As would be understood by those skilled in the art, the handle, in an exemplary embodiment, may be configured with a deployment mechanism such as a trigger grip, or any other manner suitable to deploy the closure device 104 from the internal lumen 108 of the sheath 106. The closure device 104 may be actuable by, and separable from, the delivery element 102 in any suitable manner, as described in greater detail below.

Referring to FIG. 1, a closure device 104 is shown in a first, predeployment configuration within the sheath 106, while a second closure device 104 is shown deployed within the fallopian tube 112. In an exemplary embodiment, during insertion into the body, the closure device 104 is held within the sheath 106 of the delivery element 102. The entire closure device 104 may be received within the sheath 106 or, alternatively, a part of the closure device 104, i.e. a distal end thereof, may project distally from a distal end of the sheath 106, as shown in FIG. 1. The sheath 106 may retain the closure device 104 in any suitable manner, such as by friction fit. The closure device 104 may be detachably connected to the sheath 106 by a breakable membrane, as would be understood by those skilled in the art.

The closure device 104, according to an exemplary embodiment shown in FIG. 2, is configured as a plug comprising an elongated, substantially cylindrical body 110 extending from a proximal end 107 to a distal end 109. The closure device 104 is generally circular in cross section and sized to fit snugly in the fallopian tube 112. The closure device 104 has a diameter, d, of, for example, 0.5 mm to 10 mm and a length, L, of 2 mm to 20 mm. However, it will be understood that the closure device 104 may have any other suitable diameter and length and/or any other suitable shape or profile so long as the device 104 creates a suitable seal in the fallopian tube without injuring the patient. For example, the closure device 104 may increase distally in diameter from a proximal end to a maximum diameter at a medial portion thereof before decreasing in diameter toward a distal end such that the device surface extends along a convex curve, as shown in the embodiment of FIG. 1. Alternately, the closure device 104 may be cylindrical or shaped in any other suitable manner. The closure device 104 is preferably made of a biocompatible polymer or any other suitable material. The polymer is preferably compressible and has a significant tensile strength. As one skilled in the art would understand, the polymer itself may be compressible and thus expand upon deployment, or alternatively, the polymer may be on an expandable stent-like framework (similar to a transcatheter aortic valve replacement (TAVR) valve). Suitable polymers include silicone, polyurethanes (PU), polytetrafluroethylene (PTFE), ethylene tetrafluoroethylene (ETFE), polysiobutylene polyurethane (PIB-PUR), poly(styrene-block-isobutylene-block-styrene) (SIBS), or any other suitable biocompatible material. Preferably, the closure device 104 is formulated with a conventional radiopaque material so that its presence and location can be verified by means of an X-ray.

The closure device 104 may include an atraumatic tip 114 located distal end. For example, the tip 114 may be formed as part of a hemisphere or any other suitable convex curve. In addition, the device 104 may be coated in an antibacterial coating and other surface modifiers which reduce risk of tissue in-growth (e.g. Teflon) and designed to minimize trauma to tissue with which it comes into contact as it is advanced into the fallopian tube 112. The tip 114 is, for example, 1 mm in diameter or less, such that the tip 114 can be received easily in a fallopian tube 112, an in the inner lumen of the delivery element, and in the working channel of a conventional endoscope. However, it will be understood that the tip 114 may have any other suitable diameter and/or any other suitable shape or profile so as not to damage the fallopian tube tissue.

In an exemplary embodiment, as can be seen in FIG. 2, the distal end of the closure device 104 includes a tissue grasping element 116 that may be configured in any suitable manner. As one example, the tissue grasping element 116 includes a plurality of tines 118 extending distally from a radially outer edge of the tip 114 and then curving radially outward away from a longitudinal axis of the device 104 and then extending proximally so that sharpened ends 120 of the tines 118 point proximally. This arrangement allows the device 104 to be inserted distally into a fallopian tube without resistance from the tines 118 which will fold back against the device 104 as it is advanced distally. If, later, forces later urge the device 104 proximally, the sharpened ends 120 of the tines 118 will penetrate the tissue of the fallopian tube anchoring the device 104 in place. It will be understood by those skilled in the art that the grasping element may extend from a proximal end of the device 104 or from any other location or locations along the length of the device 104 as desired. The tines 118 may be connected to one another or may be independent from one another. The tines 118 may be oriented so that their distal ends are initially positioned on or adjacent to an outer surface of the body 110, proximal to the atraumatic tip 112. In the depicted embodiment, the device 104 includes four tines 118. However, it is understood that any number of tines 118 may be used. The tissue grasping element 116 may be formed from any suitable biocompatible material, such as Nitinol. In another embodiment, the tissue grasping element 116 may include tines extending generally proximally such that the tines engage the fallopian tube 112 surface immediately upon release from the delivery element 102. The tines 118 may be connected to the body 110 of the closure device 104 in any suitable manner, such as by molding, friction fit, adhesive or welding.

In an exemplary embodiment, as can be seen in FIGS. 3-4, a closure device 104′ may include a one-way valve 130′ permitting fluid flow in a distal to proximal direction (i.e., to permit fluid to exit the fallopian tubes 112′ into the uterus 124′) while preventing fluid flow in the proximal to distal direction (i.e., to prevent fluid from moving from the uterus 124′ into the fallopian tubes 112′). In this embodiment, the device 104′ may be configured as a plug comprising an elongated, substantially cylindrical body 110′ including a lumen 126′ and a tapered proximal end 128′ having a slit valve 130′ (i.e., “duckbill valve”) open to the lumen 126′. The body 110′ may be dimensioned substantially similarly to the body of closure device 104. At a proximal end, the body 110′ tapers in the proximal direction towards the flat slit valve opening 130′ which is open to the lumen 126′ extending from the proximal end of the device 128′ to the distal end. Valve 130′ may have an exemplary slit length of approximately 1 mm. The device 104′ tapers to the proximal end 128′ having a diameter of approximately ½ of the body 110′ or less, for example. Valve 130′ may be configured such that pressure in the distal direction causes the valve 130′ to close while pressure in the proximal direction, through the lumen 126′ causes the valve 130′ to open. For example, the valve 130′ may be formed as a duck bill valve or as any known check valve. In this embodiment, device 104′ is preferably made of a biocompatible polymer or any other suitable material. The polymer is preferably compressible and has a significant tensile strength. Suitable polymers include silicone, polyurethanes (PU), polytetrafluroethylene (PTFE), ethylene tetrafluoroethylene (FIFE), polyisobutylene polyurethane (PIB-PUR), poly(styrene-block-isobutylene-block-styrene) (SIBS), or any other suitable biocompatible material. Preferably, the closure device 104 is formulated with a conventional radiopaque material so that its presence and location can be determined by means of an X-ray. Although not depicted in FIG. 3, to facilitate fixation of the device inside the fallopian tube, it is contemplated that the entire outer diameter of the device could taper form the distal end to the proximal end (where valve 110′ is located). For example, in one embodiment the distal end of the device may have an outer diameter of 3 mm and a proximal end of the device may have an outer diameter of 1 mm. This configuration would help “trap” the device in the fallopian tube such that is does not migrate out of the fallopian tube.

In an exemplary embodiment shown in FIG. 4, the device 104′ includes a proximal “cage” snaring member 132′ which facilitates removal of the device 104′. The cage member 132′ extends about and the body 110′ from a distal end 134′ to a proximal end 136′ extending about 1 to 10 mm proximally past the proximal end 137′ of the body 110′ surrounding the slit valve 130′. Because the cage member 132′ extends proximally past the device 104′, the proximally extending part of the cage member 132′ may be grasped (e.g., via an endoscopic snare, grasper, etc.) to pull the device 104′ proximally out of the fallopian tube for removal from the body. The cage member 132′ has a diameter that approximates or is slightly larger than the diameter of the body 110′ so that the cage member 132′ fits snugly about the external surface of the device 104′. The device 104′ may be connected to the body 110′ of the closure device 104′ in any suitable manner, such as by molding, friction fit, adhesive or welding so that, when the cage member 132′ is pulled proximally, the device 104′ is also pulled proximally. In an embodiment, a polymer seal (or seals) could be incorporated on the outer diameter of the cage member 132′ to ensure leakage does not occur between the cage and the fallopian tube. In another exemplary embodiment, the cage member 132′ is coupled to a proximal end of the body 110′, and extends therefrom to surround the slit valve 130′. In this embodiment, the cage member 132′ extends about a proximal portion of the body 110′ and extends proximally about 1 to 10 mm beyond the proximal end 137′ of the body 110′ in the same manner described above. In this embodiment, the cage member 132′ may be embedded into the body 110′ or otherwise firmly fixed thereto. The cage member 132′ is preferably made of wire, as shown in FIG. 4, formed of any suitable, biocompatible material. In the depicted embodiment, the cage member 132′ is configured as a cage including four connecting members 138′ and a plurality of circumferential struts 140′ disposed about the length of the body 110′. However, it will be understood that any number of connecting members 138′ and struts 140′ may be used. The cage member 132′ may include a tissue grasping element 116′ that may be configured in any suitable manner similar to the element 116 described above. In another embodiment, the cage member 132′ may be formed of Nitinol or another shape memory material to form a self-expanding cage so that, when placed in the desired position within the fallopian tube, the cage member 132′ expands to lodge firmly in the fallopian tube anchoring the device 104′ therein. As would be understood in the art, exposure to heat within the fallopian tube raises the temperature of the Nitinol cage member 132′ above a threshold temperature causing expansion from an insertion configuration to a memorized shape corresponding to the expanded configuration.

In an exemplary embodiment, as can be seen in FIGS. 7-8, a closure device 204 may be configured as an expandable stent formed of, for example, Nitinol or any other known biocompatible, shape memory material. Closure device 204 may be configured as a plug comprising an elongated, substantially cylindrical body 210 extending from a proximal end 207 to a distal end 209 including a lumen 226 extending therethrough. Body 210 may be dimensioned substantially similarly to the body of closure devices 104, 104′. Closure device 204 includes a plurality of flaps 231 extending from the proximal end 207 of the body 210. FIG. 8 shows the closure device 204 in an insertion position. Closure device 204 extends from a distal end of the sheath 206 such that flaps 231 are positioned about the exterior circumference of the sheath 206. The sheath 206 may retain the closure device 204 in any suitable manner, such as friction fit. Flaps 231 are movable from the insertion configuration to a deployed configuration shown in FIG. 8 when the sheath 206 is retracted out of the body. As shown in FIG. 7, the flaps 231 are biased to a closed configuration in which they extend toward the longitudinal axis of the closure device 204. Flaps 231 overlap one another so that pressure in one direction meets resistance while pressure in an opposing direction causes the flaps 231 to slightly open. In use, when the closure device 204 is inserted into the fallopian tubes 212, the flaps 231 permit fluid flow in a distal to proximal direction (i.e., to permit fluid to exit the fallopian tubes 212 into the uterus 224) while preventing fluid flow in the proximal direction (i.e., to prevent fluid from moving from the uterus 224 into the fallopian tubes 212). FIGS. 7-8 depict a closure device 204 including five flaps 231 however, it is understood that any number of flaps may be used. Flaps 231 may be connected to the body 210 of the closure device 204 in any suitable manner, such as by molding, friction fit, adhesive or welding.

In an embodiment, the closure/blocking devices 104, 104′, 204 may be used in conjunction with a cervix expansion device 142, which increases the cervical port diameter to avoid retrograde flow and promote flow of fluid out of the uterus through the cervix 144 and vaginal canal and helps prevent retrograde flow through the fallopian tubes 112. The expansion device 142 may be in the form of an elastic ring or band, with an opening 146 extending therethrough. Expansion device 142 is inserted into the cervix 144 such that the outer circumference of the expansion device 142 is flush against the inner circumference of the cervical wall 148, with the opening 146 aligning with the opening 150 of the cervix 144, to expand the opening 150 of the cervix 144. The expansion device 142 has an opening 146 that is greater in diameter than the diameter of the cervical opening 150 to be treated. In an exemplary embodiment, the band has a thickness, t, of about 0.5 mm an inside diameter, d, of 5 mm and an outside diameter, D, of 6 mm. The expansion device 142 can be constructed of any of various elastic materials, such as, for example, Nitinol, and is formed to exert enough outward force, F, against the walls 148 of the cervix 144 to expand the cervix opening 150 permanently after implantation. In an exemplary embodiment, the expansion device 142 may be composed of Nitinol or another shape memory material that expands from an insertion configuration to a deployed configuration upon insertion into the cervix 144. The expansion device 142 may be held within the cervix 144 in any suitable manner, such as by friction fit.

According to an exemplary method, as shown in FIG. 1, the closure/blocking device 104 and part of the sheath 106 of the delivery element 102 are advanced through the cervix into the uterus, and then into one of the fallopian tubes. In an embodiment, the device 104 and part of the sheath 106 are advanced into the fallopian tube through the working channel of an endoscope. The atraumatic tip 114 of the body 110 at the distal end of the device 104 substantially minimizes damage to the interior of the fallopian tube 112 as the device 104 is moved distally through the lumen 108. After the device 104 has been moved to the desired position within the lumen of the fallopian tube 112, motion of the device 104 stops.

Referring back to FIG. 1, the closure device 104 is in an initial position when it moves into the lumen of the fallopian tube 112. In that initial position, grasping device 116 may be deformed into a delivery configuration where the tines 118 are pressed proximally against the outer surface of the body 110. In another example, the distal rounded portion of the tines 118 may simply be folded against themselves. In another example, no grasping device 116 is used. The user then actuates the handle (not shown) of the delivery element 102. As described above, the handle may include a trigger grip, knob or any other suitable actuation mechanism. As the user actuates the handle, the closure device 104 is urged distally out of the sheath 102 of the delivery mechanism 102. As the closure device 104 leaves the sheath 106 the grasping mechanism expands outward into the deployed configuration such that the tines 118 are urged against the wall of the fallopian tube 112. The closure device 104 may be deployed such that it is fully positioned within the fallopian tube 112 or so that a portion of the closure device 104 extends into the uterus. In a preferred embodiment, the closure device 104 is retracted slightly proximally so that the tines, which are configured in a fishhook-like manner, engage and are lodged in the walls of the fallopian tube 112. Retraction of the closure device 104 can occur simply by movement of the sheath 106 proximally during withdrawal of the delivery element 102 or through an actuation device in the handle. The tines 118 may penetrate the wall of the fallopian tube 112 or may enter partially into the wall of the fallopian tube 112 but do not pass through the full thickness of the wall of the fallopian tube 112. In another example, where the device 104 does not include a grasping device 116, the device may expand from an insertion configuration to a deployed configuration, due to the shape memorization properties of the material, to friction fit within the fallopian tube 112.

The sheath 106 is separated from the closure device 104 leaving the closure device 104 in place in the fallopian tube 112. The sheath 106 may be separated from the closure device 104 in any suitable manner. As one example, the sheath 106 is friction fit or interference fit with the closure device 104 such that a known force overcomes the fit and causes the sheath 106 to separate from the closure device 104, where the known force is at least equal to the force that securely holds the closure device 104 in place in the fallopian tube 112. The sheath 106 of the delivery element 102 is then completely withdrawn from the body. The process may then be repeated in the other fallopian tube in the same manner.

If a cervical expansion device 142 is to be used, the expansion device 142 is inserted through the vaginal canal into the opening 150 of the cervix 144 via an insertion tube (not shown) or by any other suitable manner. The device 142 is implanted with the outer circumference of the expansion device 142 flush against the inner circumference of the cervical wall 148 so that the opening 146 aligns with the cervix opening 150. This outward force applied by the device 142 forces the cervix 144 to expand to the dimensions of the expansion device 142, with the opening 146 of the expansion device 142 is greater in diameter than the initial diameter of the cervix 144.

As shown in FIG. 9, a method for treating endometriosis according to another exemplary embodiment, comprises a closure device 304 in the form of an elongated string-like flexible member 352 extending from a proximal end to a distal end. In this embodiment, external blockage of the fallopian tubes 312 may be achieved by reversibly tying the flexible member 352 about the fallopian tubes 312 so that each tube 312 is compressed to a point of closure. This reversible closure of the fallopian tube 312 prevents retrograde flow from the uterus to the peritoneal cavity by completely blocking the lumen of the fallopian tube 312. The flexible closure member 352 may be formed of a flexible material such as, for example, nylon, silk, polypropylene, expanded polytetrafluroethylene (ePTFE), polyester, polyvinylindene fluoride (PVDF). The flexible member 352 may have, for example, a diameter, of less than 0.5 mm with a length suitable to extend around the entire circumference of the fallopian tube. For example, the closure member 352 may be a suture, very long in length, e.g. 16 cm, and potentially cut after securing in place around the fallopian tube. In another example, the closure member 352 may only be 5 cm in length. The flexible member 352 is delivered to the fallopian tube laparoscopically and may be tied in any suitable manner such as, for example, in a knot 354 about the fallopian tube 352. The flexible member 352 may be removed from the fallopian tube 312 in any suitable manner, such as, for example, cutting the flexible member 352 or untying the knot 354 from the fallopian tube 352.

As shown in FIG. 10, a method for treating endometriosis according to another exemplary embodiment, comprises a closure device 404 in the form of a ring of magnets 451. In this embodiment, external blockage of the fallopian tubes 412 may be achieved by reversibly placing the magnets 451 about the fallopian tubes 412 so that each tube 412 is compressed to a point of closure, similar to closure device 304. This reversible closure of the fallopian tube 412 prevents retrograde flow from the uterus to the peritoneal cavity by blocking the lumen of the fallopian tube 412. Magnets 451 attract to one another, causing closure, but still allowing fluid to pass when pressure starts to build. The device 404 as shown in FIG. 8 depicts nine magnets, however, it is understood that any number of magnets may be used. Magnets 451 may be coupled to one another to form a line of magnets 451 in any suitable manner, such as, for example, by molding, friction fit, adhesive or welding. In another example, magnets 451 may be connected via a connection member such as a string or other flexible structure. Magnets 451 may be positioned around the fallopian tube 412 laparoscopically and may be removed from the fallopian tube 412 in any suitable manner.

It will be apparent to those skilled in the art that various modifications may be made in the present disclosure without departing from the scope of the disclosure. Thus, it is intended that the present disclosure cover modifications and variations of this disclosure provided that they come within the scope of the appended claims and their equivalents.

1-15. (canceled)

16. A fallopian tube closure device, comprising:
an elongated body extending from a proximal end to a distal end and including a channel extending therethrough; and
a valve coupled to a proximal end of the body and open to the channel, the valve configured to only allow fluid to flow proximally therethrough from the channel.

17. The device of claim 16, wherein the valve is a slit valve.

18. The device of claim 16, further comprising:
a tissue grasping element coupled to a distal end of the body.

19. The device of claim 18, wherein the grasping element comprises a plurality of tines, the tines including sharpened ends configured to penetrate the wall of the fallopian tube.

20. The device of claim 16, further comprising:
a snare element extending proximally from the body and configured to allow easy removal of the closure device.

21. The device of claim 20, wherein the snare element is configured as a cage extending about the length of the body.

22. The device of claim 19, wherein the grasping element is formed of Nitinol or another shape memory material.

23. The device of claim 20, wherein the elongated body is expandable from a low-profile insertion configuration to an expanded deployed configuration.

24. The device of claim 16, further comprising:
a delivery sheath extending between a proximal end and a distal end, wherein the body is slidably received within the delivery sheath, the elongated body sized and configured to fit in the sheath in a insertion configuration.

25. A system for preventing retrograde flow through the fallopian tubes, comprising:
an elongated closure device extending from a proximal end to a distal end, the device sized and shaped to be inserted into the fallopian tube;
an expansion device including an opening extending therethrough and configured to be inserted into the cervix such that the opening aligns with a cervix opening to be treated, the opening having a diameter greater than a diameter of the cervix opening to be treated.

26. The system of claim 25, wherein the body has a channel extending therethrough.

27. The system of claim 26, further comprising:
a valve coupled to a proximal end of the body and open to the channel, the valve configured to only allow fluid to flow proximally therethrough from the channel.

28. The system of claim 25, further comprising:
a tissue grasping element coupled to a distal end of the body.

29. The system of claim 28, wherein the grasping element comprises a plurality of tines, the tines including sharpened ends configured to penetrate the wall of the fallopian tube.

30. The system of claim 25, further comprising:
a snare element extending proximally from the body and configured to allow easy removal of the closure device.

31. A method for closing a fallopian tube, comprising:
inserting a closure device into the fallopian tube, the closure device extending from a proximal end to a distal end and including a channel extending therethrough; and
securing the closure device to the fallopian tube via one of friction fit and a grasping element,
wherein the closure device further comprises a valve coupled to a proximal end thereof and open to the channel, the valve configured to only allow fluid to flow proximally therethrough from the channel.

32. The method of claim 30, wherein the grasping element comprises a plurality of tines, the tines including a sharpened end configured to penetrate the wall of the fallopian tube.

33. The method of claim 31, further comprising:
inserting a flexible delivery sheath into the lumen of the fallopian tube, the delivery sheath extending between a proximal end and a distal end and including a channel extending therethrough,
wherein the closure device is sized and shaped to fit within the sheath in a collapse configuration.

34. The method of claim 32, further comprising:
withdrawing the delivery sheath from the fallopian tube, wherein proximal movement of the delivery sheath moves the closure device proximally so that the grasping element penetrates a wall of the fallopian tube.

35. The method of claim 30, wherein the closure device includes a snare element extending proximally from the body and configured to allow easy removal of the closure device.
